(19) United States
(12) Patent Application Publication
(10) Pub. No.: US 2024/0279161 A1
(43) Pub. Date: Aug. 22, 2024

(54) AZIDO-CONTAINING MONOMERS, POLYMERS, AND ARTICLES

(71) Applicant: 3M INNOVATIVE PROPERTIES COMPANY, St. Paul, MN (US)

(72) Inventors: Brylee David B. Tiu, Minneapolis, MN (US); Stephen B. Roscoe, Woodbury, MN (US); Daniel J. Schmidt, Woodbury, MN (US); Joshua M. Fishman, St. Louis Park, MN (US)

(21) Appl. No.: 18/566,689

(22) PCT Filed: May 19, 2022
(86) PCT No.: PCT/IB2022/054699

§ 371 Date: Dec. 4, 2023

Related U.S. Application Data
(60) Provisional application No. 63/202,547, filed on Jun. 16, 2021.

(51) Int. Cl. C07C 241/04; C08F 220/34; C08J 7/04

(52) U.S. Cl. CPC C07C 241/04; C08F 220/34; C08J 7/042; C08J 2333/14; C08J 2379/02

(57) ABSTRACT

An azido-containing monomer, a polymer containing monomeric units derived from the azido-containing monomer, and various articles containing a substrate and a coating layer of the polymer positioned on the substrate are provided. The azido groups, which are pendant groups of the polymer, can undergo a click chemistry reaction with an unsaturated compound such as those having a terminal —C≡CH group, a dibenzocyclooctyne-containing group, an unsaturated bicyclic olefinic group, or an amido group. The click chemistry reaction can be used to covalently attach a fluorescent or bioactive compound to the polymer.

### BACKGROUND

Various methods are known for attaching compounds to a surface. Additional methods are desired.

### SUMMARY

An azido-containing monomer, a polymer containing monomeric units derived from the azido-containing monomer, and various articles containing a substrate and a coating layer of the azido-containing polymer positioned on the substrate are provided. The azido groups in the polymer can undergo a click chemistry reaction with an unsaturated compound such that the unsaturated compound is covalently linked to the polymer. The unsaturated compound can have, for example, a carbon-carbon triple bond, an unsaturated bicyclic olefinic group, or an acrylamido group. The unsaturated compound can be, for example, a fluorescent compound or a bioactive material.

In a first aspect, an azido-containing monomer of Formula (I) is provided.

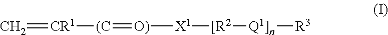

In Formula (I), the group R¹ is hydrogen or methyl and the group X¹ is —NH— or —O—. The variable n is equal to 0 or 1, the group R² is a (hetero)alkylene having at least one oxygen heteroatom (i.e., R² is an ether or polyether group), group Q¹ is —(C═O)—X²— or —NH—(C═O)—X²—, and the group X² is —NH— or —O—. Group R³ is either (a) an ether or polyether group terminated with an azido group or (b) a branched hetero-hydrocarbon group terminated with two or more azido groups, the hetero-hydrocarbon group having a branching location and comprising (i) an ether or polyether group before the branching location and (ii) an ether or polyether group in each branch after the branching location.

In a second aspect, a polymer is provided that contains a monomeric unit derived from the azido-containing monomer of Formula (I) described in the first aspect. The polymer can be a homopolymer or a copolymer.

In a third aspect, a first article is provided. The first article comprises a substrate and a coating positioned on the substrate. The coating comprises one or more layers of different polymers, wherein at least one layer comprises a first polymer comprising a first monomeric unit derived form an azido-containing monomer of Formula (I) as described above in the first aspect.

In some embodiments of the third aspect, the coating comprises one or more bilayers comprising a first layer and a second layer adjacent to the first layer. The first layer comprises the first polymer comprising first monomeric units derived from the azido-containing monomer of Formula (I) while the second layer comprises a second polymer having a monomeric unit that interacts with the first polymer electrostatically or through hydrogen bonding. Either the first layer or the second layer is adjacent to the substrate.

In a fourth aspect, a second article is provided. The second article comprises a reaction product of the first article, which is described above in the third aspect, with an unsaturated compound capable of undergoing a click chemistry reaction with an azido group of the first monomeric unit in the first polymer, wherein the unsaturated compound is covalently linked to the first polymer.

The terms “a”, “an”, and “the” are used interchangeably and mean one or more.

The term “and/or” means to one or both. For example, the expression X and/or Y means X alone, Y alone, or both X and Y.

The term “azido” refers to a monovalent group —N₃.

The term “alkyl” refers to a monovalent group that is a radical of an alkane and includes groups that are linear, branched, cyclic, bicyclic, or a combination thereof Unless otherwise indicated, the alkyl groups typically contain from 1 to 20 carbon atoms. In some embodiments, the alkyl groups contain 1 to 10 carbon atoms, 2 to 10 carbon atoms, 1 to 6 carbon atoms, 2 to 6 carbon atoms, 1 to 4 carbon atoms, or 2 to 4 carbon atoms. Cyclic alkyl groups and branched alkyl groups have at least three carbon atoms. Examples of alkyl groups include, but are not limited to, methyl, ethyl, n-propyl, n-butyl, n-pentyl, isobutyl, t-butyl, isopropyl, n-octyl, n-heptyl, ethylhexyl, cyclopentyl, cyclohexyl, cycloheptyl, adamantyl, norbornyl, and the like.

The term “alkylene” refers to a divalent group that is a radical of an alkane and includes groups that are linear, branched, cyclic, bicyclic, or a combination thereof. Unless otherwise indicated, the alkylene groups typically contain from 2 to 20 carbon atoms. In some embodiments, the alkyl groups contain 2 to 10 carbon atoms, 2 to 8 carbon atoms, 2 to 6 carbon atoms, 2 to 4 carbon atoms, 3 carbon atoms, or 2 carbon atoms. Cyclic alkyl groups and branched alkyl groups have at least three carbon atoms. Examples of alkylene groups include, but are not limited to, methylene, ethylene, n-propylene, n-butylene, n-pentylene, isobutylene, t-butylene, isopropylene, n-octylene, n-heptylene, ethylhexylene, cyclopentylene, cyclohexylene, cycloheptylene, adamantylene, norbornylene, and the like.

The term “heteroalkylene” refers to alkylene where at least one carbon atom between two other carbon atoms is replaced with a heteroatom. The heteroatom is typically oxygen, nitrogen, or sulfur. The heteroalkylene often has one or more oxygen heteroatoms. In some examples, the heteroalkylene contains multiple ethylene groups or propylene groups separated by oxygen heteroatoms.

The term “(hetero)alkylene” refers to an alkylene, heteroalkylene, or both.

The term “hydrocarbon” refers to refers to a compound or group having only carbon and hydrogen atoms.

The term “hydrogen bond donor” refers to a group that has at least one hydrogen atom that is supplied to form a hydrogen bond. Typically, a more electroactive atom such as oxygen or nitrogen is bonded to the hydrogen atom.

The term “hydrogen bond acceptor” refers to a group that can accept at least one hydrogen atom to form a hydrogen bond. The hydrogen bond acceptor often has at least one lone pair of electrons.

The term “hetero-hydrocarbon” refers to a compound or group having carbon, hydrogen, and heteroatoms. Heteroatoms typically include nitrogen, oxygen, and sulfur.

The term “ether group” refers to a group having an oxygen atom between two alkylene groups.

The term “polyether group” is a heteroalkylene having multiple oxygen heteroatoms. The heteroalkylene contains multiple ethylene groups or propylene groups separated by oxygen heteroatoms.

The term “monomeric unit” refers to a polymerized product of a monomer. For example, the monomeric unit associated with the monomer acrylic acid (H₂C═CH—(C═O)—OH) is

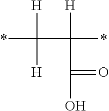 [SMILES: *CC(*)C(=O)O]

where the asterisk (*) is an attachment site to another monomeric unit or terminal group in the polymer.

The term “pendant group” refers to a group that is not part of the backbone of the polymer. For example, in a polymer containing monomeric unit derived from acrylic acid, the pendant group is —(C═O)—OH.

Dashes on either side of groups such as —O— and —NH— indicate that these groups are divalent. A dash on a single side of a group such as —(C═O)—OH indicates that this group is monovalent.

The term “polymer” includes homopolymers, copolymers, terpolymers, and the like. The term “copolymer” is used herein to include any polymer having at least two different types of monomeric units.

The term “click chemistry” refers to the reaction of an unsaturated compound with an azido-containing compound to form a cyclic group having three nitrogen atoms and covalently linking the unsaturated compound to the azido-containing compound. The unsaturated compound typically has a terminal —C═CH group, a carbon-carbon triple bond in a cyclic group such as a cyclic group with eight ring members, a carbon-carbon double bond in a bicyclic olefinic group such as in norbornene, or an acrylamido group with a carbon-carbon double bond conjugated to a —(C═O)—NH— group. These types of reactions are also referred to as Huisgen reactions.

The term “bioactive molecule” refers to a compound that can interact with a biological system or on a living organism. The compound can be synthetic or biologically derived and can have any desired molecular weight. Examples include, but are not limited to, nucleic acid-containing compounds (e.g., oligonucleotides and polynucleotides), amino acid-containing compounds (e.g., peptides, oligopeptides, and polypeptides including proteins and antibodies), carbohydrate-containing compounds (e.g. monosaccharides, oligosaccharides, and polysaccharides), biotin, lipids, pharmaceutical compounds, secondary metabolites, cofactor for enzymes, vitamins, hormones, synthetic analogues of any of these species and the like and mixtures thereof.

### DETAILED DESCRIPTION

An azido-containing monomer and an azido-containing polymer having monomeric units derived from the azido-containing monomer are provided. Further, various articles containing the azido-containing polymer or containing the click chemistry reaction product of the azido-containing polymer are provided. The articles typically include a substrate and a coating positioned on the substrate. The coating can be a single layer or can contain one or more bilayers comprising a first layer adjacent to a second layer that interacts with the first layer by electrostatic and/or hydrogen bonds. The first layer contains the azido-containing polymer but either the first layer or the second layer can be positioned adjacent to the substrate.

### Azido-Containing Monomers

In a first aspect, an azido-containing monomer of Formula (I) is provided.

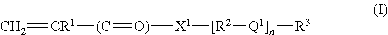

In Formula (I), the group R¹ is hydrogen or methyl and the group X¹ is —NH— or —O—. The variable n is equal to 0 or 1, the group R² is an alkylene or a heteroalkylene having at least one oxygen heteroatom (i.e., ether or polyether group), group Q¹ is —(C═O)—X²— or —NH—(C═O)—X²—, and the group X² is —NH— or —O—. Group R³ is either (a) an ether or polyether group terminated with an azido group or (b) a branched hetero-hydrocarbon group terminated with two or more azido groups, the hetero-hydrocarbon group having a branching location and comprising (i) an ether or polyether group before the branching location and (ii) an ether or polyether group in each branch after the branching location.

In Formula (I), the variable n is equal to 0 to 1. When the variable n is equal to 1, the monomer of Formula (I) is of Formula (I-1). When the variable n is equal to 0, the monomer of Formula (I) is of Formula (I-2).

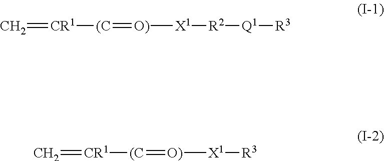

When R¹ is hydrogen, the azido-containing monomer has a polymerizable group that is either an acryloyloxy group (CH₂═CH—(C═O)—O—) when X¹ is —O— or an acryloylamido group (CH₂═CH—(C═O)—NH— when X¹ is —NH—. When R¹ is methyl, the azido-containing monomer has a polymerizable group that is either a methacryloyloxy group (CH₂═C(CH₃)—(C═O)—O—) when X¹ is —O— or methacryloylamido group (CH₂═C(CH₃)—(C═O)—NH—) when X¹ is —NH—.

In Formulas (I) and (I-1), the group R² is a (hetero)alkylene. If R² is an alkylene, it often has 1 to 10, 1 to 6, 2 to 6, 2 to 4, 3, or 2 carbon atoms. The heteroalkylene group can have 1 to 5 oxygen heteroatoms and often contains 2 to 10 carbon atoms. In some embodiments, the alkylene is —CH₂CH₂—, —CH₂CH₂CH₂—, or —C(CH₃)₂— and the heteroalkylene is —(CH₂—CH₂—O)ₓ—CH₂CH₂— or —(CH₂—CH₂—CH₂—O)ₓ—CH₂CH₂—CH₂— where x is 1, 2, or 3.

In Formulas (I) and (I-1), the group Q¹ can be —(C═O)—X²— or —NH—(C═O)—X²— with X² being —O— or —NH—. That is, Q¹ can be —(C═O)—O—, —(C═O)—NH—, or —NH—(C═O)—O—, or —NH—(C═O)—NH—.

Group R³ in any of the above Formulas (I), (I-1) and (I-2) is either (a) an ether or polyether group terminated with an azido group or (b) a branched hetero-hydrocarbon group terminated with two or more azido groups, the hetero-hydrocarbon group having a branching location and comprising (i) an ether or polyether group before the branching location and (ii) an ether or polyether group in each branch after the branching location. Thus, R³ typically contains at least one azido group. In many embodiments, group R³ contains 1 or 2 azido groups.

Example R³ groups that are an ether or polyether group terminated with an azido group are often of formula —R⁴—(O—R⁴)ₘ—O—R⁴—N₃ where each R⁴ is an alkylene and m is an integer in a range of 0 to 40. In some embodiments, each R⁴ is either ethylene or propylene. The variable m can be 0, at least 1, at least 2, at least 3, at least 4, at least 5, at least 6, at least 8, or at least 10 and up to 40, up to 36, up to 32, up to 30, up to 28, up to 24, up to 20, up to 16, up to 12, up to 10, up to 8, up to 6, or up to 4.

Other example R³ groups have two or more azido groups and include a branching group. Some branched R³ groups are of formula —R⁵—O—(R⁵—O)ₚ—R⁵—N[—R⁵—O—(R⁵—O)_{q}—R⁵—N₃]₂ where each R⁵ is independently an alkylene, p is an integer in a range of 0 to 30 and q is an integer in a range of 0 to 10. The alkylene R⁵ usually has 1 to 4 carbon atoms and is often either ethylene or propylene. The variable p is equal to at least 0, at least 1, at least 2, at least 4, or at least 10 and up to 30, up to 24, up to 20, up to 18, up to 16, up to 14, up to 12, or up to 10. The variable q is equal to at least 0, at least 1, at least 2, at least 3, or at least 4 and up to 10, up to 8, up to 5, or up to 4.

Other branched R³ groups are of formula —R⁶—O—(R⁶—O)ₓ—R⁶—(C═O)—NH—CH[—R⁶—O—R⁶—(C═O)—NH—R⁶—O—(R⁶—O)_{y}—R⁶—N₃]₂ where each R⁶ is independently an alkylene, x is in integer in a range of 0 to 30, and y is an integer in a range of 0 to 10. The alkylene R⁶ usually has 1 to 4 carbon atoms and is often either ethylene or propylene. The variable x is equal to at least 0, at least 1, at least 2, at least 4, or at least 10 and up to 30, up to 24, up to 20, up to 18, up to 16, up to 14, up to 12, or up to 10. The variable y is equal to at least 0, at least 1, at least 2, at least 3, or at least 4 and up to 10, up to 8, up to 5, or up to 4.

In some embodiments of Formulas (I) and (I-1), X¹ is equal to —O— and Q¹ is equal to —NH—(C═O)—X²— where X² is either —NH— or —O—. The azido-containing monomer is of Formula (I-A).

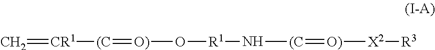

The azido-containing monomers of Formula (I-A) can be formed, for example, by reaction of an isocyanato-containing monomer with an azido-containing compound having a hydroxy or —NH₂ group that can react with the isocyanato group. The azido-containing compound is typically of formula HX²—R³ where X² and R³ is the same as defined above. This reaction is shown in Reaction Scheme A.

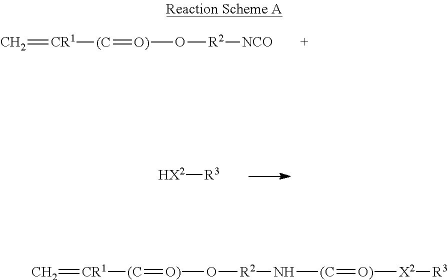

Some example compounds of monomers of formula CH₂═CR¹—(C═O)—O—R²—NCO include, but are not limited to, CH₂═CR¹—(C═O)—O—CH₂CH₂—NCO, CH₂═CR¹—(C═O)—O—CH₂CH₂CH₂—NCO, and CH₂═CR¹—(C═O)—O—(CH₂—CH₂—O)ₓ—CH₂CH₂—NCO where x is equal to 1, 2 or 3. The group R¹ is the same as described above for Formula (I).

Compounds of HX²—R³ can be of two different types. In the first type, R³ is an ether or polyether group terminated with an azido group and X² is —O— or —NH—. In the second type, R³ is a branched hetero-hydrocarbon group terminated with two or more azido groups, the hetero-hydrocarbon group having a branching location and comprising (i) an ether or polyether group before the branching location and (ii) an ether or polyether group in each branch after the branching location.

Examples of the first type of HX²—R³ compounds are of formula HX²—R⁴—(O—R⁴)ₘ—R⁴—N₃ where X² is —O— or —NH—, each R⁴ is an alkylene and m is an integer in a range of 0 to 40. Some more specific examples include, but are not limited to, H₂N—CH₂CH₂—(O—CH₂CH₂)ₘ O—CH₂CH₂—N₃ and HO—CH₂CH₂—(O—CH₂CH₂)ₘ—O—CH₂CH₂—N₃ where m ranges from 0 to 40.

Some examples of the second type of HX²—R³ compounds are of formula HX²—R⁵—O—(R⁵—O)ₚ—R⁵—N[—R⁵—O—(R⁵—O)_{q}—R⁵—N₃]₂ where X² is —O— or —NH—, each R⁵ is independently an alkylene, p is an integer in a range of 0 to 30, and q is an integer in a range of 0 to 10. The alkylene R⁵ usually has 1 to 4 carbon atoms and is often either ethylene or propylene. More specific examples include, but are not limited to, compound of formula H₂N—CH₂CH₂—O—(CH₂CH₂—O)ₚ—CH₂CH₂—N[CH₂CH₂—O—(CH₂CH₂—O)_{q}—CH₂CH₂—N₃]₂ where p is an integer in a range of 0 to 30 and q is an integer in a range of 0 to 10.

Other examples of HX²—R³ compounds of the second type are of formula HX²—R⁶—O—(R⁶—O)ₓ—R⁶—(C═O)—NH—CH[—R⁶—O—R⁶—(C═O)—NH—R⁶—O—(R⁶—O)_{y}—R⁶—N₃]z where X² is —O— or —NH—, each R⁶ is independently an alkylene, x is in integer in a range of 0 to 30 and y is an integer in a range of 0 to 10. The alkylene R⁶ usually has 1 to 4 carbon atoms and is often either ethylene or propylene. More specific examples include, but are not limited to, compound of formula H₂N—CH₂CH₂—O—(CH₂CH₂—O)ₓ—CH₂CH₂—(C═O)—NH—CH[CH₂CH₂—O—CH₂CH₂—(C═O)—NH— CH₂CH₂—O—(CH₂CH₂—O)_{y}—CH₂CH₂—N₃]z where x is in integer in a range of 0 to 30 and y is an integer in a range of 0 to 10.

In other embodiments of Formulas (I) and (I-1), X¹ is —NH—, Q¹ is —(CO)—X²—, and X² is —O or —NH—. The azido-containing monomer is of Formula (I-B).

CH₂═CR¹—(C═O)—NH—R²—(C═O)—X²—R³

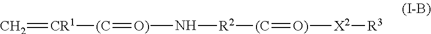

In many embodiments, R¹ is hydrogen and R² is an alkylene such as —C(CH₃)₂—. That is, the azido-containing monomer of Formula (I-B) is often CH₂═CH—(C═O)—NH—C(CH₃)₂—(C═O)—X²—R³.

The azido-containing monomers of Formula (I-B) such as those of formula CH₂═CH—(C═O)—NH—C(CH₃)₂—(C═O)—X²—R³ can be formed by reaction of vinyl azlactone with an azido-containing compound having a hydroxy or —NH₂ group that can react with the azlactone group as shown in Reaction Scheme B. The azido-containing compound is typically of formula HX²—R³ as described above for use in Reaction Scheme A.

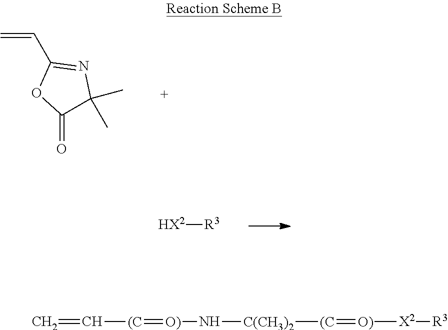

Although the azido-containing monomer is usually of Formula (I-1), it can be of Formula (I-2) as described above

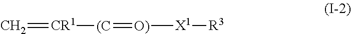

Monomers of Formula (I-2) can be formed, for example, by reaction of (meth)acrylate anhydride with an azido-containing compound of formula HX²—R³ as described above. That is, both X¹ and X² are either hydrogen or methyl. This reaction is shown in Reaction Scheme C. The group X¹ in Formula (I-2) is the same as group X² in the compound HX²—R³.

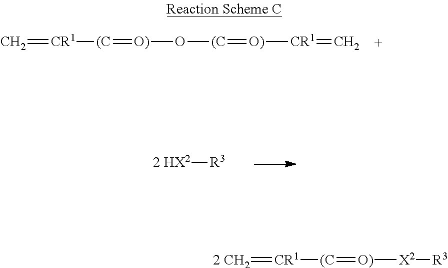

### Azido-Containing Polymers

Any of the above described azido-containing monomers can be polymerized to form a polymer. The polymer can be a homopolymer, copolymer, terpolymer, or the like.

The azido-containing monomer can be polymerized with a second monomer having an ethylenically unsaturated group. Any known second monomer having an ethylenically unsaturated group can be used. For example, the second monomer can be a (meth)acrylate monomer, a (meth)acrylamide monomer, a vinyl monomer, or an allyl monomer. In many embodiments, the second monomer is a (meth)acrylate or (meth)acrylamide monomer.

In some embodiments, the azido-containing monomer is polymerized with a second monomer that can form electrostatic bonds or hydrogen bonds with another polymeric chain. The second monomer can have an anionic group, a cationic group, a hydrogen bond acceptor group, a hydrogen bond donor group, or a combination thereof. Other monomers that do not have any of these groups can also be used, if desired.

Second monomers that have an acidic group (i.e., acidic monomers) can be an anionic group or can provide a hydrogen bond donor group depending on the pH. Some acidic groups such as —(C═O)—OH can function as either a hydrogen donor group or even as a hydrogen acceptor group through the carbonyl group.

Some second monomers that have an amino group can be a cationic monomer or can provide a hydrogen bond acceptor group depending on the pH. Example amino groups include primary amino group, secondary amino group, tertiary amino group, quaternary amino group, or a salt thereof. Suitable salts include halides, acetate, sulfates, phosphates, hydroxides, and the like.

Many other second monomers can be selected that provide either a hydrogen bond donor, a hydrogen bond acceptor, or both. Monomers that contain a carbonyloxy group —(C═O)—O— such as in various esters, an ether group, or a carbonyl group that is not attached to either oxygen or nitrogen (such as in a ketone) can function as hydrogen bond acceptor groups. Other monomers with a hydroxyl group (—OH) (such as those not bonded to a carbonyl), —NH group (such as those not bonded to a carbonyl), —(C═O)—NH— group, —(C═O)—OH group, —(C═O)—NH— group can function as hydrogen bond donating groups or hydrogen bond acceptor groups.

Second monomers that are acidic monomers can have a carboxylic acid group (—COOH), a phosphoric acid group (—O—PO₃H₂), a phosphoric acid ester group (—O—PO₃RH where R is hydrogen or alkyl), a phosphonic acid group (—PO₃H₂), a phosphonic acid ester group (—PO₃RH where R is hydrogen or alkyl), sulfonic acid group (—SO₃H), or a salt thereof. The counter cation for the salt can be any suitable cation such as alkaline metal cations, alkaline earth metal cations, transition metal cations, quaternary ammonium cations, and the like.

Example acidic monomers include, but are not limited to, (meth)acrylic acid, β-carboxyethyl (meth)acylate, 2-(meth)acryloyloxyethyl phthalic acid, 2-(meth)acryloyloxy succinic acid, vinyl phosphoric acid, phosphoric acid 2-hydroxyethyl (meth)acrylate ester, vinyl phosphonic acid, phosphonoalkyl (meth)acrylate, vinyl sulfonic acid, vinyl hydrogen sulfate, styrene sulfonic acid, and 2-acrylamido-2-methylpropane sulfonic acid, 2-sulfoethyl (meth)acrylate, or salts thereof. Salts of acidic monomers include, but are not limited to, zinc (meth)acrylate, zirconium (meth)acrylate, zirconium carboxyethyl acrylate, and the like.

Second monomers with an amino group that have a primary amino group, secondary amino group, tertiary amino group, quaternary amino group, or a salt thereof. Suitable salts include halides, acetate, sulfates, phosphates, hydroxides, and the like.

Primary amino-containing second monomers include, but are not limited to, vinyl amine, allyl amine, aminoalkyl(meth)acrylamide (e.g., 2-aminoethyl (meth)acrylamide and 3-aminopropyl (meth)acrylamide), aminoalkyl (meth)acrylate (e.g., 2-aminoethyl (meth)acrylate and 3-aminopropyl (meth)acrylate), 2-N-morpholinoalkyl (meth)acrylate, and hydrochloride salts thereof.

Secondary amino-containing second monomers include, but are not limited to, various alkylaminoalkylene (meth)acrylates such as, for example, 2-(methylamino)ethyl (meth)acylate and salts thereof.

Tertiary amino-containing second monomers include, but are not limited to, various N,N-dialkylaminoalkyl (meth)acrylates and N,N-dialkylaminoalkyl (meth)acrylamides such as N,N-dimethyl aminoethyl (meth)acrylate, N,N-dimethylaminoethyl (meth)acrylamide, N,N-dimethylaminopropyl (meth)acrylate, N,N-dimethylaminopropyl (meth)acrylamide, N,N-diethylaminoethyl (meth)acrylate, N,N-diethylaminoethyl (meth)acrylamide, N,N-diethylaminopropyl (meth)acrylate, and N,N-diethylaminopropyl (meth)acrylamide, (tert-butylamino)alkyl (meth)acrylate (e.g., tert-butylaminoethyl (meth)acrylate and tert-butylaminopropyl (meth)acrylate), (tert-butylamino)alkyl (meth)acrylamide (e.g., tert-butylaminoethyl (meth)acrylate and tert-butylaminopropyl (meth)acrylamide), and salts thereof.

Quaternary amino-containing monomers include, but are not limited to, methacryloylaminopropyl trimethylammonium chloride, diallyldimethylammonium chloride, and 2-acryloxyalkyltrimethylammonium chloride.

Second monomers that have a hydroxyl group include, but are not limited to, hydroxy-substituted alkyl (meth)acrylates and hydroxy-substituted alkyl (meth)acrylamides. Specific examples include 2-hydroxyethyl (meth)acrylate, 2-hydroxypropyl (meth)acrylate, 3-hydroxypropyl (meth)acrylate, 4-hydroxybutyl (meth)acylate, 2-hydroxyethyl (meth)acrylamide, 2-hydroxypropyl (meth)acrylamide, 3-hydroxypropyl (meth)acrylate, and 4-hydroxybutyl (meth)acrylamide, and the like.

Second monomers that have an ether group include, but are not limited to, vinyl methyl ether, 2-methoxyethoxyethyl (meth)acrylate, 2-ethoxyethoxyethyl (meth)acrylate, di(ethylene glycol)-2-ethylhexyl-ether (meth)acrylate, ethylene glycol-methyl ether (meth)acrylate, polyethylene glycol (meth)acrylate, and polypropylene glycol (meth)acrylate. The polyethylene glycol (meth)acrylate and polypropylene glycol (meth)acrylate can optionally be terminated with a hydroxy group.

Second monomers that have a —(CO)—O— group include, but are not limited to, esters of (meth)acrylic acid or vinyl esters. In many embodiments, the second monomer with a —(CO)—O— group is an alkyl (meth)acrylate having an alkyl group with 1 to 20 carbon atoms, 1 to 18 carbon atoms, 1 to 12 carbon atoms, 1 to 10 carbon atoms, 1 to 6 carbon atoms, or 1 to 4 carbon atoms. Examples include, but are not limited to, methyl (meth)acrylate, ethyl (meth)acrylate, n-propyl (meth)acrylate, iso-propyl (meth)acrylate, n-butyl (meth)acrylate, sec-butyl (meth)acrylate, isobutyl (meth)acrylate, n-pentyl (meth)acrylate, 2-methylbutyl (meth)acrylate, n-hexyl (meth)acrylate, cyclohexyl (meth)acrylate, 4-methyl-2-pentyl (meth)acrylate, n-octyl (meth)acrylate, 2-ethylhexyl (meth)acrylate, n-octyl (meth)acrylate, iso-octyl (meth)acrylate, 2-octyl (meth)acrylate, 3,3,5 trimethylcyclohexyl (meth)acrylate, n-nonyl (meth)acrylate, isoamyl (meth)acrylate, isobornyl (meth)acylate, n-decyl (meth)acylate, isodecyl (meth)acrylate, n-decyl (meth)acrylate, lauryl (meth)acrylate, isotridecyl (meth)acrylate, n-octadecyl (meth)acrylate, stearyl (meth)acrylate, isostearyl (meth)acrylate, n-dodecyl methacrylate, and combinations thereof.

Second monomers that have a —NH— group such as —(C═O)—NH— include, but are not limited to, (meth)acrylamide as well as various N-alkyl (meth)acrylamides and N-alkoxyalkyl (meth)acrylamides such as N-methyl (meth)acrylamide, N-ethyl (meth)acrylamide, N-isopropyl (meth)acrylamide, N-propyl(meth)acrylamide, N-tert-butyl(meth)acrylamide, N-(triphenylmethyl)(meth)acrylamide, N-phenyl(meth)acrylamide, N-(4-hydroxyphenyl)(meth)acrylamide and N-octyl (meth)acrylamide, N-(3-methoxypropyl)acrylamide, and N-(isobutoxymethyl)acrylamide. Others include N-vinyl carbazole, N-vinyl caprolactam, N-vinyl-2-pyrrolidone, N-vinyl azlactone, 4-(meth)acryloylmorpholine, N-vinylimidazole, ureido (meth)acrylate, and N,N-dialkyl (meth)acrylamides such as N,N-dimethyl (meth)acrylamide, N,N-diethyl (meth)acrylamide, N,N-dipropyl (meth)acrylamide, or N,N-dibutyl (meth)acrylamide.

The amount of the azido-containing monomer of Formula (I) used to form the polymer can be any desired amount ranging from 0.1 to 100 mole percent based on total moles of monomeric units in the polymer. The amount can be at least 0.1 mole percent at least 0.5 mole percent, at least 1 mole percent, at least 2 mole percent, at least 5 mole percent, at least 10 mole percent, at least 15 mole percent, at least 20 mole percent, at least 25 mole percent, at least 30 mole percent, at least 35 mole percent, or at least 40 mole percent and up to 100 mole percent, up to 90 mole percent, up to 80 mole percent, up to 70 mole percent, up to 60 mole percent, or up to 50 mole percent. The monomeric units that do not contain an azido group can be derived from one or more of the second monomers described above or from any other known monomer.

In some embodiments, the polymer contains 0.1 to 50 mole percent monomeric units derived from the azido-containing monomer of Formula (I) and 50 to 99.9 mole percent monomeric units derived from any second monomer such as those described above. For example, the polymer can contain 0.5 to 50 mole percent, 1 to 50 mole percent, 0.1 to 40 mole percent, 0.5 to 40 mole percent, 1 to 40 mole percent, 2 to 40 mole percent, 5 to 40 mole percent, 0.1 to 30 mole percent, 0.5 to 30 mole percent, 1 to 30 mole percent, 2 to 30 mole percent, 5 to 30 mole percent, 0.1 to 20 mole percent, 0.5 to 20 mole percent, 1 to 20 mole percent, 2 to 20 mole percent, 5 to 20 mole percent, 0.1 to 10 mole percent, 0.5 to 10 mole percent, 1 to 10 mole percent, 0.1 to 5 mole percent, 0.5 to 5 mole percent, or 1 to 5 mole percent of the azido-containing monomer of Formula (I) with the remainder comprising one or more second monomers described above.

The azido-containing polymer often has a weight average molecular weight in a range of 1000 to 500,000 grams/mole (Daltons, Da). For example, the weight average molecular weight can be at least 1000 Da, at least 2000 Da, at least 5000 Da, at least 10,000 Da, at last 20,000 Da, at least 30,000 Da, or at least 50,000 Da and up to 500,000 Da, up to 450,000 Da, up to 400,000 Da, up to 300,000 Da, up to 200,000 Da, up to 100,000 Da, or up to 50,000 Da. The weight average molecular weight can be determined using Gel Permeation Chromatography.

### Articles Containing Azido-Containing Polymers

In a third aspect, articles are provided that include a substrate and a coating that contains the azido-containing polymer positioned on the substrate. The azido-containing polymer is any of those described above. The coating layer containing the azido-containing polymer can be positioned adjacent to the substrate or can be separated from the substrate by one or more other coating layers.

In some articles, the coating contains multiple layers and the azido-containing polymer can be in one or more of these multiple layers. For example, the coating can contain one or more bilayers. Each bilayer has a first layer adjacent to a second layer with the first layer and second layers interacting with each other by electrostatic bonds and/or hydrogen bonds.

As used herein, the “first polymer” is in a “first layer” of the bilayer and the “second polymer” is in a “second layer”. The first polymer in the first layer comprises a monomeric unit derived from an azido-containing monomer of Formula (I) as described above. The second polymer in the second layer may optionally comprise a monomeric unit derived from an azido-containing monomer of Formula (I). Either the first layer or the second layer of the bilayer can be positioned adjacent to the substate. If there are multiple bilayers, the bilayers are arranged so that each layer interacts by electrostatic and/or hydrogen bond interactions with any adjacent layer.

When the bilayers interact by electrostatic interactions, either (1) the first layer contains a first polymer that is a polyanionic polymer and the second layer contains a second polymer that is a polycationic polymer or (2) the first layer contains a first polymer that is a polycationic polymer and the second layer contains a second polymer that is a polyanionic polymer. That is, both the first and second layers contain a polymer with ionic groups. The polycationic polymer, the polyanionic polymer, or both may contain the azido-containing monomer of Formula (I).

In some bilayer examples based on electrostatic interactions, the bilayer includes a first polymer that is a polyanionic polymer and that contains monomeric units derived from the azido-containing monomer of Formula (I). The second polymer in the bilayer is a polycationic polymer. In these examples, the first polymer that is a polyanionic polymer is prepared from a mixture of the azido-containing monomer of Formula (I) and a second monomer that is an acidic monomer such as those that contain a carboxylic acid group (—COOH), a phosphoric acid group (—O—PO₃H₂), a phosphoric acid ester group (—O—PO₃RH where R is hydrogen or alkyl), a phosphonic acid group (—PO₃H₂), a phosphonic acid ester group (—PO₃RH where R is hydrogen or alkyl), sulfonic acid group (—SO₃H), or a salt thereof. Such monomers are described above. Any suitable polycationic polymer can be used as the second polymer.

Some example second polymers that are polycationic polymers can be prepared from monomers that can have a positive charge depending on the pH. Suitable monomers usually have an amino group such as a primary amino group, secondary amino group, tertiary amino group, quaternary amino group, or a salt thereof. Suitable salts include halides, nitrates, acetate, sulfates, phosphates, hydroxides, and the like. Example amino-containing monomers that can be used to prepare polycationic polymers are described above. Although other types of monomers can be copolymerized with the amino-containing monomers that can have a positive charge, the amounts of these other types of monomers is often low (e.g., less than 5 weight percent, less than 1 weight percent, less than 0.5 weight percent, or less than 0.1 weight percent) to maximize the electrostatic interactions between adjacent layers in the bilayer.

Some specific examples of second polymers that can be polycationic and that are prepared by free radical polymerization include, but are not limited to, poly(allylamine hydrochloride), polyvinylamine, poly(vinylbenzyltrimethylammonium chloride), polydiallyldimethylammonium chloride, poly(dimethylaminoethyl methacrylate), and poly(methacryloylamino)propyltrimethylammonium chloride. Other example second polymers that are or can be polycationic polymers with a pH adjustment include, but are not limited to, linear and branched poly(ethylenimine) (PEI), chitosan, polyaniline, or polyamidoamine.

In a specific example of bilayers that interact by electrostatic interactions, the first polymer contains monomeric units of the azido-containing monomer of Formula (I) and a second monomer that has a carboxylic acid group or salt thereof while the second polymer is PEI.

In other bilayer examples based on electrostatic interactions, the bilayer includes a first polymer that is a polycationic polymer having monomeric units derived from the azido-containing monomer of Formula (I). The second polymer in the bilayer is a polyanionic polymer. In these examples, the first polymer is prepared from a mixture of the azido-containing monomer of Formula (I) and a second monomer that can have a positive charge depending on the pH. Suitable second monomers usually have a primary amino group, secondary amino group, tertiary amino group, quaternary amino group, or a salt thereof. Such monomers are described above. Any suitable polyanionic polymer can be used as the second polymer.

Some example polyanionic polymers that can be used as the second polymer are prepared by free radical polymerization of acidic monomers such as those described above that contain a carboxylic acid group (—COOH), a phosphoric acid group (—O—PO₃H₂), a phosphoric acid ester group (—O—PO₃RH where R is hydrogen or alkyl), a phosphonic acid group (—PO₃H₂), a phosphonic acid ester group (—PO₃RH where R is hydrogen or alkyl), sulfonic acid group (—SO₃H), or a salt thereof. Some such polymers include, for example, poly(vinyl sulfuric acid), poly(vinyl sulfonic), poly(acrylic acid) (PAA), poly(methacrylic acid) (PMA), poly(styrene sulfonic acid). Other example polyanionic polymers include, for example, dextran sulfate, heparin, hyaluronic acid, carrageenan, carboxymethylcellulose, alginate, and sulfonated tetrafluoroethylene-based fluoropolymers such as those available under the trade designation NAFION.

In other bilayer examples based on electrostatic interactions, both the first polymer and the second polymer contain monomeric units derived from the azido-containing monomer of Formula (I). In these examples, the first polymer is usually prepared from a mixture of the azido-containing monomer of Formula (I) and a second monomer that is an acidic monomer such as those that contain a carboxylic acid group (—COOH), a phosphoric acid group (—O—PO₃H₂), a phosphoric acid ester group (—O—PO₃RH where R is hydrogen or alkyl), a phosphonic acid group (—PO₃H₂), a phosphonic acid ester group (—PO₃RH where R is hydrogen or alkyl), sulfonic acid group (—SO₃H), or a salt thereof. Such monomers are described above. The second polymer is prepared from a mixture of the azido-containing monomer of Formula (I) and a second monomer that can have a positive charge depending on the pH. Suitable second monomers usually have a primary amino group, secondary amino group, tertiary amino group, quaternary amino group, or a salt thereof. Such monomers are described above.

In other bilayer embodiments, the first layer and the second layer interact by hydrogen bonding. One layer contains a hydrogen bond donor and the other layer contains a hydrogen bond acceptor. The first polymer included in the first layer contains the azido-containing monomer of Formula (I) and the second polymer included in the second layer may optionally contain the azido-containing monomer of Formula (I). When the bilayers interact by hydrogen bonding, either (1) the first layer contains a first polymer that has hydrogen bond donor groups and the second layer contains a second polymer that has hydrogen bond accepting groups or (2) the first layer contains a first polymer that has hydrogen bond accepting groups and the second layer contains a second polymer has hydrogen bond donor groups. Both polymers are neutral rather than ionic.

In some bilayer examples based on hydrogen bonding, the first layer contains a first polymer having monomeric units derived from the azido-containing monomer of Formula (I) and second monomeric units having hydrogen bond donor groups. Second monomers with hydrogen bond donor groups are often acidic monomers such as those described above. Any suitable second polymer having hydrogen bond acceptor groups can be used in the second layer. Monomers with hydrogen bond acceptor groups often contain an atom having an available electron pair such as, for example, a hydroxy group (—OH), an ether group, a carbonyl group such as in a nitrogen-containing ring, or a carbonyl-containing group such as —(CO)—O— or —(CO)—NH—. Such monomers are described above. In some embodiments, the second polymer is polyacrylamide, poly(ethylene oxide), poly(N-vinyl pyrrolidone), poly(N-isopropyl acrylamide), poly(vinyl methyl ether), poly(N-vinyl caprolactam), or poly(2-hydroxyethyl acrylate).

In other bilayer examples based on hydrogen bonding, the first layer contains a first polymer having monomeric units derived from the azido-containing monomer of Formula (I) and second monomeric units having hydrogen bond acceptor groups. Second monomers with hydrogen bond acceptor groups often contain often contain an atom having an available electron pair such as, for example, a hydroxy group (—OH), an ether group, a carbonyl group such as in a nitrogen-containing ring, or a carbonyl-containing group such as —(CO)—O— or —(CO)—NH—. Such monomers are described above. Any suitable second polymer having hydrogen bond donor groups can be used in the second layer. Monomers with hydrogen bond donor groups are often acidic monomers such as those described above (the pH is such that the acidic monomers are not ionized). Some such polymers include, for example, poly(vinyl sulfuric acid), poly(vinylsulfonic acid), poly(acrylic acid) (PAA), poly(methacrylic acid) (PMA), and poly(styrene sulfonic acid). Other example polymers with hydrogen bond donor groups include, for example, dextran sulfate, heparin, hyaluronic acid, carrageenan, carboxymethylcellulose, alginate, and sulfonated tetrafluoroethylene-based fluoropolymers such as those available under the trade designation NAFION. Poly(vinyl alcohol) can also be used as a hydrogen bond donor polymer.

In still other bilayer examples based on hydrogen bonding, both the first polymer in the first layer and the second polymer in the second layer contain monomeric units derived from the azido-containing monomer of Formula (I). The first polymer contains second monomeric units with hydrogen bond donor groups while the second polymer contains second monomeric units with hydrogen bond acceptor groups. Suitable second monomers with hydrogen bond donor groups and with hydrogen bond donor groups are described above.

The articles can have any desired number of bilayers. In some embodiments, the number of bilayers can be at least 1, at least 2, at least 3, at least 5 and up to 10 or more, up to 8, up to 6, up to 4, or up to 3.

In any of the articles, any suitable substrate can be used. The substrate can be a polymeric material, glass, ceramic material, glass ceramic, silicon, metallic material, metal oxide, and the like. Suitable polymeric materials include, but are not limited to, polyesters (e.g., polyethylene terephthalate and polyethylene naphtholate), polycarbonate, polyvinyl chloride, polystyrene, polyurethane, polyether sulfone, various (meth)acrylic resins including polymethyl methacrylate, cellulosic materials, polyolefins (e.g., polypropylene, polyethylene and polymers containing cyclic olefins such as cyclic olefin polymers (e.g. ARTON sold by Japan Synthetic Rubber and ZEONOR or ZEONEX sold by Zeon Chemical), cyclic olefin copolymers (e.g. TOPAS sold by Topas Advanced Polymers and APEL sold by Mitsui Chemicals) and cyclic block copolymers (e.g. VIVION sold by USI Corporation)), polyamides (e.g. NYLON 6 and NYLON 6,6), polyetheretherketones (e.g. KETASPIRE sold by Solvay), polyimides (e.g. KAPTON sold by DuPont), and copolymers thereof.

If the layers will interact by electrostatic attraction, the surface of the substrate may be selected or treated so that it has ionic groups. If the layers will interact through hydrogen bonds, the surface of the substrate may be selected or treated so that is has hydrogen bond acceptor group or hydrogen bond donor groups. Various treatment options can be used to provide the desired functional groups, if necessary. For example, polyolefins can be subjected to corona, plasma, UV ozone, or caustic treatments to provide a negatively charged surface. Such surfaces are well suited for deposition of bilayers that interact electrostatically. Alternatively, a substrate can be treated with a polycation as an initial adhesive layer or optionally further treated with a layer of a polyanion. The polyanion surface can be used for deposition of bilayers that interact by hydrogen bonding with the layer nearest the polyanion surface containing hydrogen bond acceptor groups.

Deposition of the first bilayer or of multiple bilayers often involves exposing a substrate with a suitable surface to a series of liquid solutions or baths. This can be done by immersion of the substrate into liquid baths (also referred to as dip coating), spraying, spin coating, roll coating, inkjet printing, or the like. After each immersion, the excess liquid is typically removed by washing. Each successive liquid has characteristics that will result in electrostatic or hydrogen bonding interactions between adjacent layers. That is, the plurality of layers making up the coating are deposited by layer-by-layer (LBL) self-assembly.

The liquid solutions that are used to construct the bilayers typically contains polymer dissolved in water or a water miscible solvent such as an alcohol, glycol, and the like. Any desired concentration of the polymer can be used provided it is soluble in the liquid.

If the bilayers interact by electrostatic bonding, the substrate can be treated or selected to have an ionic group. The first coating composition is selected to contain a first polyionic polymer with an ionic charge opposite to that of the substrate. When the substrate is exposed to the first coating composition, the ionic species on the surface of the substate are attracted to the oppositely charged first polyionic polymer in the first coating. Adsorption occurs until the substrate surface is covered with first polyionic polymer. The absorption process can vary from a few seconds to a few minutes. The polyionic polymer masks the surface of the substrate and provides an outer surface with a charge opposite to that of the substrate.

After adsorption of the first polyionic polymer, the coated substrate is washed to remove any excess first polyionic polymer and then coated with a second polyionic polymer. The charge of the second polyionic polymer is opposite that of the first polyionic polymer. Adsorption occurs until the first polyionic polymer is covered with second polyionic polymer. The absorption process can vary from a few seconds to a few minutes. The second polyionic polymer masks the first polyionic polymer and provides an outer surface with a charge opposite to that of the first polyionic polymer.

After adsorption of the second polyionic polymer, the coated substrate is washed to remove any excess second polyionic polymer. Further coating layers can be applied that alternate in between those containing polycationic and polyanionic polymers.

Similar processing steps can be used to apply bilayer that interact by hydrogen bonding rather than by electrostatic interactions. One layer contains a polymer with hydrogen bond donor groups and the adjacent layer contains a polymer with hydrogen bond acceptor groups.

If desired, the bilayers can be crosslinked. Any suitable crosslinking method can be used. In some embodiments, groups in adjacent layers within the bilayer can react with each other. For example, monomeric units containing amine groups in one layer can react with carboxylic acid groups in an adjacent layer to form an amido linkage (—(C═O)—NH—). This reaction typically requires exposure to heat (about 200 degrees Celsius). Milder conditions can be used, however, by adding a coupling agent that can convert the carboxylic acid groups (—COOH) into acid chlorides, anhydrides, or active esters. Examples of coupling agents include N-hydroxysuccinimide (NHS), dicyclohexylcarbodiimide (DCC), and 1-ethyl-3-(3-dimethylaminopropyl)carbodiimide (EDC). Additionally, amine-containing polymers can be crosslinked using glutaraldehyde.

Others methods of crosslinking include reacting a polymer with amine (—NH₂) or hydroxyl (—OH) groups with a monomer such as glycidyl (meth)acrylate or 2-hydroxyethyl (meth)acrylate to introduce pendant (meth)acryloyl groups that can be crosslinked using ultraviolet radiation. Other suitable crosslinking methods are further described, for example, in U.S. Pat. No. 9,393,589 (Olmeijer et al.). Photo-crosslinkable monomers such as (meth)acrylic monomers functionalized with benzophenone, cinnamoyl, or coumarin can be included in the polymers used in the bilayers and these groups can result in crosslinking when exposed to ultraviolet radiation.

The first articles having one or more bilayer have many advantages. For example, the layer-by-layer coatings can be prepared from water-based compositions and have little or no volatile organic compounds. The coatings are conformable and can be applied to many different types of substrates. Further, the layer-by-layer coatings offer accessibility to the functional groups in the bulk of the coating due to swelling. Still further, the coatings do not need to be covalently bonded to the substrate. Additionally, the thickness of the coatings can be finely tuned based on the number of bilayers deposited ranging from a nanometer to hundreds of nanometers thick.

The articles described above (i.e., the first articles) that include at least one layer with a polymer containing monomeric units derived from the azido-containing monomer of Formula (I) can be reacted with an unsaturated compound capable of undergoing a click chemistry reaction with an azido group. Such compounds often have a terminal —C≡CH group, a carbon-carbon triple bond (—C≡C—) such as in a cyclic group having eight ring members, a carbon-carbon double bond (—C≡C—) as in an unsaturated bicyclic olefinic group having seven ring members, or an acrylamido group. The click chemistry reaction results in the covalent attachment of the unsaturated compound to the polymer.

There are numerous commercially available compounds having a group that can undergo a click chemistry reaction with an azido group. Additionally, there are several companies that specialize in adding a group capable of undergoing a click chemistry reaction to a specifically selected compound such a polynucleotide (e.g., oligonucleotide) or a polypeptide (e.g., oligopeptide), carbohydrates (e.g. monosaccharides, oligosaccharides or polysaccharides), or other bioactive molecules (e.g. biotin). Such companies include, for example, JPT Peptide Technology (Berlin, Germany) Integrated DNA Technologies (Coralville, IA, USA), ThermoFisher Scientific (Waltham, MA, USA), Jena Biosciences (Jena, Germany), CarboSynth Ltd (Berkshire, United Kingdom), and BroadPharm (San Diego, CA, USA).

Further, there are several companies that provide fluorescent dyes having a carbon-carbon triple bond. These include fluorescent dyes commercially available under the trade designation AFDye (e.g., AFDye 350 alkyne and AFDye 488 DBCO), Cy5 alkyne, Oregon Green 488 alkyne, and TAMRA alkyne from Click Chemistry Tools (Scottsdale, AZ, USA). Similar fluorescent dyes are commercially available from ThermoFisher Scientific (Waltham, MA, USA), BroadPharm (San Diego, CA, USA), and Conju-Probe, LLC (San Diego, CA, USA).

For compounds having a terminal carbon-carbon triple bond (—C≡CH), the click chemistry reaction with the polymer having azido-containing groups is shown schematically in Reaction Scheme D.

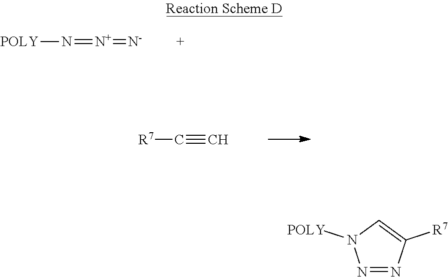

For ease of discussion, the polymer (POLY) is shown having only one azido group but, as described above, the polymer typically contains 0.1 to 50 mole percent monomeric units derived from the azido-containing monomer of Formula (I). Group R⁷ group is the residual of the carbon-carbon triple-bond containing compound minus the group —C≡CH. That is, the reaction results in covalent attached of the group R⁷ to the polymer.

Any suitable R⁷—C≡CH compound can be used. In some embodiments, the compound is of formula HC≡C—R—C≡CH with two terminal —C≡CH groups. Some examples of compounds having a terminal —C≡CH group include, for example, propargyl alcohol, 3-butyn-1-ol, 4-pentyn-1-ol, 5-hexyn-1-ol, hex-5-yn-1-yl acrylate, glycidyl propargyl ether, propargyl-N-hydroxysuccinimidyl ester (i.e., propargyl-NHS ester), propargyl-PEG4-NHS ester, N-propargyl maleimide, glycidyl propargyl ether, propargyl-PEG4 maleimide, propargylamine, 4,7,10,13,16-pentaoxononadeca-1,18-diyne, and 1,7-octadiyne, and dipropargylamine.

Thus, in some embodiments, the second article is a reaction product of the first article with an unsaturated compound of formula R⁷—C≡CH having a terminal —C≡CH group. The second article contains a substrate and a coating layer that includes a polymer with a plurality of groups of Formula (II)

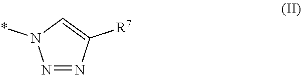

where group R⁷ group is the residual of the carbon-carbon triple bond containing compound minus the terminal —C≡CH group. The group R⁷ is often selected to increase polarity of the unsaturated compound so that it is miscible with water. The asterisk (*) is an attachment site to the polymer. More specifically, the second article contains a polymer having a plurality of monomeric units of Formula (III).

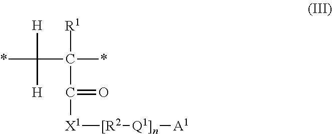

In Formula (III), groups X¹, R², and Q¹ plus the variable n are the same as defined above for Formula (I) and A¹ is the group of Formula (II). The asterisks show the attachment sites to other monomeric units in the polymer. The coating layer containing the polymer with monomeric units of Formula (III) can be positioned adjacent to the substrate or can be separated from the substrate by one or more other coating layers.

As an alternative, the unsaturated compound can have a carbon-carbon triple bond (—C≡C—) in the ring. The ring is often cyclooctyne, bicyclononyne, or a dibenzocyclooctyne-containing compound (DBCO-containing compound or DIBO-containing compound). Examples of DBCO-containing compounds and DIBO-containing compounds are of Formulas (IV-A) and (IV-B) respectively.

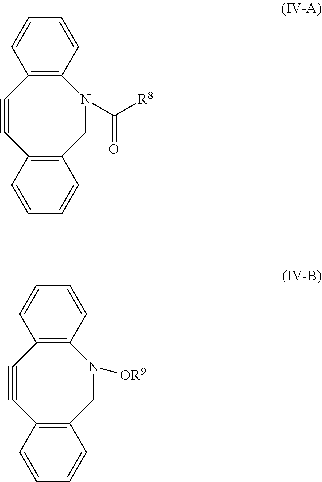

Groups R⁸ and R⁹ are typically each a polar group that increase the miscibility of the unsaturated compound with water. Various compounds of Formula (IV-A) include DBCO-amine, DBCO-C2-alcohol, DBCO-acid, DBCO-C6 acid, DBCO-NHS, DBCO-C6-NHS ester, DBCO-maleimide, DBCO-PEG3-alcohol, DBCO-PEG3-aldehyde, DBCO-PEG3-amine, DBCO-PEG3-biotin, DBCO-PEG3-oxyamine, DBCO-PEG3-NHS, DBCO-PEG4-PFP ester, sulfo DBCO-PEG3-acid, DBCO-NHCO-PEG4-acid, and methyltetrazine-DBCO. Similar polar substituents (R⁹ groups) are also available for the DIBO-containing compounds of Formula (IV-B).

For unsaturated compounds of Formula (IV-A), the click chemistry reaction with the polymer having azido-containing groups is shown schematically in Reaction Scheme E.

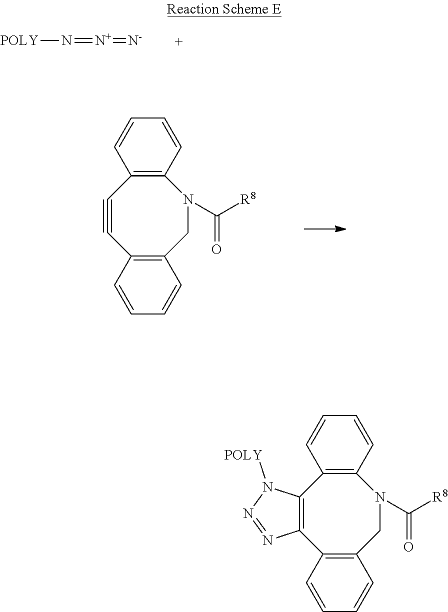

For ease of discussion, the polymer (POLY) is shown having only one azido group but, as described above, the polymer typically contains 0.1 to 50 mole percent monomeric units derived from the azido-containing monomer of Formula (I). The second article contains a substrate and a coating layer that includes a polymer with a plurality of groups of Formula (V) where R⁸ is a polar group.

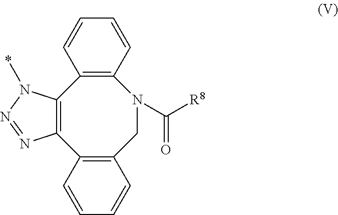

More specifically, the second article contains a polymer having a plurality of monomeric units of Formula (VI).

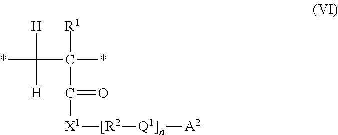

In Formula (VI), groups X¹, R², and Q¹ plus the variable n are the same as defined above for Formula (I) and A² is the group of Formula (V). The asterisks show the attachment sites to other monomeric units in the polymer. The coating layer containing the polymer with monomeric units of Formula (VI) can be positioned adjacent to the substrate or can be separated from the substrate by one or more other coating layers.

As another alternative, the unsaturated compound has a carbon-carbon double bond (—C≡C—) as in an unsaturated bicyclic olefinic group having seven ring members. The unsaturated compound is often a norbornene derivative of Formula (VII).

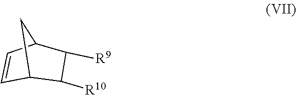

In Formula (VII), the groups R⁹ is usually a polar group that increases the miscibility of the unsaturated compound with water and group R¹⁰ is hydrogen, a polar group, or combines with R⁹ to form a ring (e.g., a nitrogen-containing ring) that can be further substituted with a polar group. Examples of unsaturated compounds of Formula (VII) include, but are not limited to, 5-norbornene-2 methanol, 5-norbornene-2 carboxylic acid, 5-norbornene-2-NHS ester, methyl 5-norbornene-2 carboxylate, 5-norbornene-2 acrylic acid, 5-norbornene-2,3 dimethanol, cis-5-norbornene-endo-2,3-dicarboxylic acid, N-(2-ethylhexyl)-5-norbornene-2,3-dicarboximide, N-hydroxy-5-norbornene-2,3-dicarboximide perfluoro-1-butanesulfonate, and N-hydroxy-5-norbornene-2,3-dicarboxylic acid imide.

For unsaturated compounds of Formula (VII), the click chemistry reaction with the polymer having azido-containing groups is shown schematically in Reaction Scheme F.

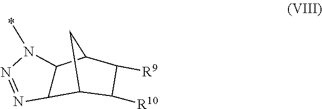

For ease of discussion, the polymer (POLY) is shown having only one azido group but, as described above, the polymer typically contains 0.1 to 50 mole percent monomeric units derived from the azido-containing monomer of Formula (I). The second article contains a substrate and a coating layer that includes a polymer with a plurality of groups of Formula (VIII) where R⁹ and R¹⁰ are defined above.

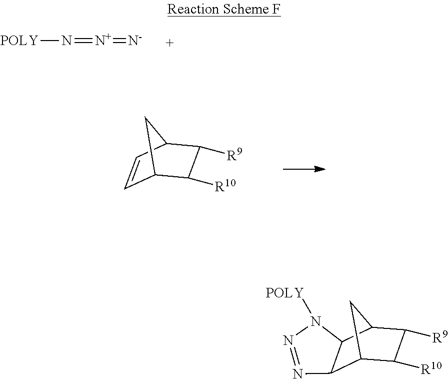

More specifically, the second article contains a polymer having a plurality of monomeric units of Formula (IX).

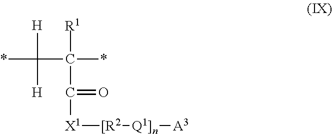

In Formula (IX), groups X¹, R², and Q¹ plus the variable n are the same as defined above for Formula (I) and A³ is the group of Formula (VIII). The asterisks show the attachment sites to other monomeric units in the polymer. The coating layer containing the polymer with monomeric units of Formula (IX) can be positioned adjacent to the substrate or can be separated from the substrate by one or more other coating layers.

In yet another alternative, the unsaturated compound has a carbon-carbon double bond (—C≡C—) conjugated to a —(C═O)—NH— group as in an acrylamido-containing compound. The unsaturated compound is often an acrylamide derivative of Formula (X).

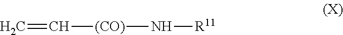

In Formula (X), group R¹¹ is (hetero)hydrocarbyl group.

For unsaturated compounds of Formula (X), the click chemistry reaction with the polymer having azido-containing groups is shown schematically in Reaction Scheme G.

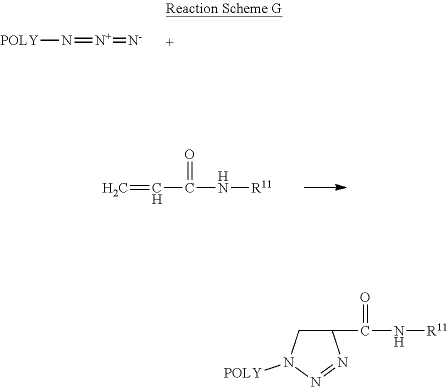

For ease of discussion, the polymer (POLY) is shown having only one azido group but, as described above, the polymer typically contains 0.1 to 50 mole percent monomeric units derived from the azido-containing monomer of Formula (I). The second article contains a substrate and a coating layer that includes a polymer with a plurality of groups of Formula (X1) where R¹¹ is defined above.

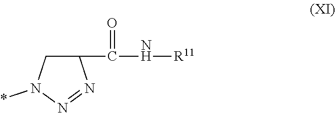

More specifically, the second article contains a polymer having a plurality of monomeric units of Formula (XII).

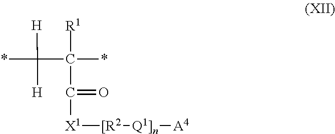

In Formula (XII), groups X¹, R², and Q¹ plus the variable n are the same as defined above for Formula (I) and A⁴ is the group of Formula (XI). The asterisks show the attachment sites to other monomeric units in the polymer. The coating layer containing the polymer with monomeric units of Formula (XII) can be positioned adjacent to the substrate or can be separated from the substrate by one or more other coating layers.

### Examples

### Materials

Copper sulfate pentahydrate, N,N-diisopropylethylamine, pyranine, and propargyl bromide (8.98 M in toluene) were obtained from Alfa Aesar, Ward Hill, MA.

5′-Hexynyl-GCG CTG TTC ATT CGC-Fluorescein-3′ was obtained from Integrated DNA Technologies, Coralville, IA.

11-Azido-3,6,9-trioxaundecan-1-amine and N-isopropylhydroxylamine hydrochloride were obtained from TCI America, Portland, OR.

Azido-PEG4-alcohol (CAS #86770-67-4), azido-PEG4-amine (CAS #951671-92-4), and N-(amino-PEG2)-N-bis(PEG3-azide) (Product #BP24369) were obtained from BroadPharm, San Diego, CA.

Ammonium persulfate was obtained from VWR Scientific, Radnor, PA.

Isocyanatoethoxyethyl methacrylate (IEEM) and isocyanatoethyl methacrylate (IEM) were obtained from Showa Denko, Europe GmbH, Munich, Germany.

Branched poly(ethylenimine) (PEI), average MW 25,000 Da, was obtained from BASF SE, Ludwigshafen, Germany under the trade designation LUPASOL WF.

Deionized water was purified using a MILLI-Q water purification system (EMD Millipore, Burlington, MA).

Methyl t-butyl ether (MTBE) was obtained from EMD Millipore, Burlington, Massachusetts.

Acrylamide, acryloyl chloride, potassium phosphate monobasic, potassium phosphate dibasic trihydrate, azobisisobutyronitrile (AIBN), N,N,N′,N″,N″-pentamethyldiethylenetriamine (PMDETA), glutaraldehyde solution (50 wt. % in water), and tetramethylethylenediamine (TMEDA) were obtained from the Sigma-Aldrich Company, St. Louis, MO.

1-(3-Dimethylaminopropyl)-3-ethylcarbodiimide hydrochloride (ED C) was obtained from Oakwood Chemical, Estill, SC.

Potassium phosphate buffer (10 mM, pH 7.0) was prepared as a solution of potassium phosphate monobasic (0.524 g) and potassium phosphate dibasic trihydrate (1.403 g) in 98.07 mL of deionized water.

4,4-Dimethyl-2-vinyl-4H-oxazol-5-one (VDM, CAS #29513-26-6) can be prepared as described in U.S. Pat. No. 4,777,276 (Rasmussen) and is also available from Isochem North America, LLC, Syosset, New York.

Silicon wafers (<100>orientation, N-type, P-dopant, 500 micron thickness, test grade) were obtained from University Wafer Incorporated, South Boston, MA.

Liquid Chromatography-Mass Spectroscopy (LCMS) data was recorded on an Agilent 1260 Infinity HPLC system with a 6130 quadrupole LC/MS (Agilent Technologies, Santa Clara, CA). Samples were chromatographed with an InfinityLab Poroshell 120 EC-C8 column using 9.8-98% acetonitrile in 6 mM ammonium formate as eluent.

Gel Permeation Chromatography (GPC), also known as Size Exclusion Chromatography (SEC) for water-soluble polymers, is a standard method for determining the molecular weights of polymers. Any commercially available GPC systems can be used such as those from Agilent Technologies (Santa Clara, CA, USA). Typical GPC elution solvents include organic solvents (e.g., tetrahydrofuran, toluene, chloroform), mixed organic water solvents (e.g. N,N-dimethylformamide\water, N-methyl-2-pyrrolidine\water), mixed water\methanol solvents, and water. The water can contain dissolved salts to provide a desired pH (e.g., the water can contain 0.2 M NaNO₃ and 0.01 M NaH₂PO₄ for a pH 7 eluent). GPC stationary phase columns may be matched to the elution solvent. Hydrophilic polymers of the current invention may be preferably characterized by eluting with mixed organic/water solvents such as N,N-dimethylformamide\water or acetonitrile\water over hydrophilic chromatography columns such as PL aquagel-OH or Polargel. Suitable detectors for GPC chromatography systems include differential refractive index (DRI) detectors, ultraviolet detectors, and light scattering detectors. The GPC system is preferably calibrated with molecular weight standards comprising polyethylene glycol/oxide (e.g. EasiVial calibration kits) using a linear least squares analysis fit to establish a calibration curve. The weight average molecular weight (Mw), the number average molecular weight (Mn) and the polydispersity index (Mw/Mn) may be determined for each polymer using the calibration curve.

### Preparatory Example 1. Synthesis of trisodium 8-prop-2-ynoxypyrene-1,3,6-trisulfonate

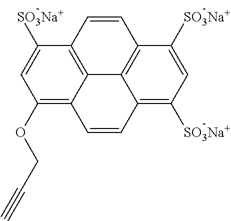

A 250 mL round bottom flask equipped with a condenser was charged with pyranine (2.6 g), N,N-diisopropylethylamine (2.0 g), methanol (150 mL), and propargyl bromide (8.98 M in toluene, 5.0 g) under an atmosphere of nitrogen. The reaction was heated to 60° C. for 16 hours. After cooling, the reaction was poured into 1.6 L of acetone and filtered to provide 2.03 g of trisodium 8-prop-2-ynoxypyrene-1,3,6-trisulfonate as a yellow solid.

### Example 1. Synthesis of Monomer A

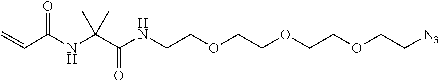 [SMILES: C=CC(=O)NC(C)(C)C(=O)NCCOCCOCCOCCN=[N+]=[N-]]

4,4-Dimethyl-2-vinyl-4H-oxazol-5-one (1.0 mL) was added to a flask containing a solution of 11-azido-3,6,9-trioxaundecan-1-amine (1.0 mL) in diethyl ether (5.0 mL, EM Science, Gardena, CA). After one hour, the upper phase was removed and diethyl ether (10 mL) added to the flask. The resulting mixture was stirred overnight and then concentrated under reduced pressure overnight to provide Monomer A as a clear oil. The product was confirmed by liquid chromatography-mass spectrometry (LCMS) analysis: 358.1 (MH⁺).

### Example 2. Synthesis of Monomer B

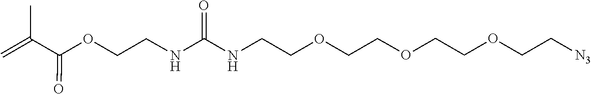 [SMILES: C=C(C)C(=O)OCCNC(=O)NCCOCCOCCOCCN=[N+]=[N-]]

Isocyanatoethyl methacrylate (IEM, 0.150 mL) was added to a flask containing a stirred solution of 11-azido-3,6,9-trioxaundecan-1-amine (0.200 mL) in diethyl ether (5 mL). An additional portion of 11-azido-3,6,9-trioxaundecan-1-amine (0.200 mL) was added to the flask, followed by an additional portion of IEM (0.150 mL). The mixture was stirred for 15 minutes and then the diethyl ether layer was removed by decanting. Fresh diethyl ether (5 mL) was added and the mixture was stirred for 30 minutes. Next, the flask was chilled in a refrigerator overnight. The bulk of the diethyl ether was decanted from the flask and any residual diethyl ether was removed under reduced pressure. The resulting Monomer B product was dissolved in 1.8 mL of N,N-dimethylformamide (DMF) to provide a solution of about 25 percent by weight (wt. %) Monomer B.

### Example 3. Synthesis of Monomer C

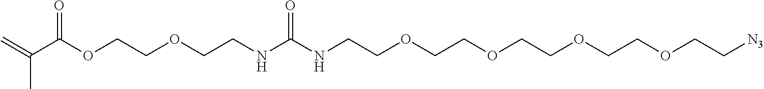 [SMILES: C=C(C)C(=O)OCCOCCNC(=O)NCCOCCOCCOCCOCCN=[N+]=[N-]]

Azido-PEG-4-amine (0.200 mL) was added with stirring to a flask containing diethyl ether (2 mL), followed by the addition of isocyanatoethoxyethyl methacrylate (IEEM, 0.160 mL). An additional portion of azido-PEG-4-amine (0.200 mL) was added to the flask, followed by an additional portion of IEEM (0.160 mL). The mixture was stirred for 45 minutes and then the diethyl ether layer was removed by decanting. Fresh diethyl ether (5 mL) was added and the mixture was stirred for 30 minutes. Next, the flask was chilled in a refrigerator overnight. Monomer C was recovered by decanting the bulk of the diethyl ether and then removing any residual diethyl ether using a gentle stream of air. The resulting Monomer C product was confirmed by LCMS analysis: 462.1 (MH⁺)

### Example 4. Synthesis of Monomer D

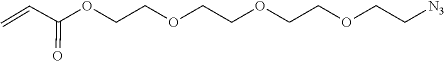 [SMILES: C=CC(=O)OCCOCCOCCOCCN=[N+]=[N-]]

Acryloyl chloride (0.19 mL) was added to a flask containing a stirred solution of azido-PEG4-alcohol (0.4555 g) in MTBE (10 mL). Diisopropylethylamine (0.4 mL) was then added and a precipitate started to form almost immediately. The reaction mixture was stirred for one hour. The solution was decanted and then concentrated under reduced pressure to provide Monomer D as a yellow oil.

### Example 5. Synthesis of Monomer E

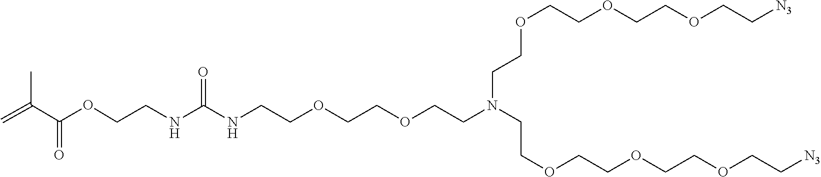 [SMILES: C=C(C)C(=O)OCCNC(=O)NCCOCCOCCN(CCOCCOCCOCCN=[N+]=[N-])CCOCCOCCOCCN=[N+]=[N-]]

Isocyanatoethyl methacrylate (IEM, 0.051 mL) was added to a flask containing N-(amino-PEG2)-N-bis(PEG3-azide) (0.050 g) in diethyl ether (0.5 mL) and the mixture was stirred for 45 minutes. The diethyl ether was decanted from the flask. Next, fresh diethyl ether (5 mL) added to the flask and the mixture was stirred for 30 minutes. The flask was chilled in a refrigerator overnight. The bulk of the diethyl ether was decanted from the flask and any residual diethyl ether was removed using a gentle stream of air. The resulting Monomer E product was confirmed by LCMS analysis: 706.4 (MH⁺).

### Example 6. Polymer-A1 (Prepared by Copolymerization of Acrylamide and Monomer A)

Acrylamide (400 mg) was added to a vial containing a solution of Monomer A (99 mg) in deionized water (5 mL). The solution was further diluted with 15 mL of deionized water and then degassed for 15 minutes by bubbling a stream of nitrogen gas into the solution. TMEDA (30 microliters) and a 5 wt. % solution of ammonium persulfate in deionized water (0.25 mL) were sequentially added to the vial. The vial was sealed and gently agitated for 1.5 hours. The polymerization reaction was quenched with isopropyl alcohol (5 mL) and then lyophilized overnight to provide Polymer-A1 as a white solid.

### Example 7. Polymer-B 1 (Prepared by Copolymerization of Acrylamide and Monomer B)

A 25 wt. % solution of Monomer B in DMF was added to a vial containing a stirred solution of acrylamide (404 mg) in deionized water (2.5 mL). The resulting solution was diluted to 25 mL with deionized water and then degassed by bubbling a stream of nitrogen gas through the solution for 15 minutes. Next, 30 microliters of TMEDA was added to the flask followed by a 5 wt. % aqueous solution of ammonium persulfate (0.25 mL). The vial was sealed and stirred at 45° C. for two hours.

The reaction was quenched by adding a 15 wt. % solution of N-isopropylhydroxylamine hydrochloride in deionized water (0.15 mL) to the flask, followed by bubbling air through the solution for ten minutes. The solution was lyophilized. The resulting solid was sequentially redissolved in water, precipitated using methanol, and collected by centrifugation. Residual solvent was removed under reduced pressure to provide Polymer-B 1 as a white solid.

### Example 8. Polymer-A2 (Prepared by Copolymerization of Acrylic Acid and 1 Mol % Monomer A)

In a flame-dried 100 mL round bottom flask with a magnetic stir bar, acrylic acid (1.9 mL) and a 25 wt. % solution of Monomer A in DMF (0.4 mL) were stirred in 84 mL of dry DMF. AIBN (46 mg) was dissolved in DMF (1 mL) and added to the flask using a syringe. A stream of nitrogen gas was bubbled through the resulting solution for 15 minutes. The flask was warmed in an oil bath heated to 60° C. and the reaction was stirred overnight. The polymerization reaction was quenched by placing the flask in a liquid nitrogen bath. The Polymer-A2 product was then precipitated by adding diethyl ether and subsequently collected by centrifugation. The collected polymer was dissolved in isopropyl alcohol, reprecipitated by adding diethyl ether, and then collected by centrifugation. Polymer-A2 was submitted to a second reprecipitation procedure and the resulting product was dried under reduced pressure overnight. Infrared spectroscopy analysis of Polymer-A2 showed an azide peak at 2100 cm⁻¹ and amide peaks at 1630 cm⁻¹ and 1530 cm⁻¹.

### Example 9. Polymer-A3 (Prepared by Copolymerization of Acrylic Acid and 3 Mol % Monomer A)

The same procedure as described in Example 8 was followed with the exception that 0.8 mL of the 25 wt. % solution of Monomer A in DMF, 1.24 mL of acrylic acid, and 31 mg of AIBN were used to prepare the copolymer. Infrared spectroscopy analysis of the Polymer-A3 product showed an azide peak at 2100 cm⁻¹ and amide peaks at 1630 cm⁻¹ and 1530 cm⁻¹.

### Example 10. Polymer-A4 (Prepared by Copolymerization of Acrylic Acid and 10 Mol % Monomer A)

The same procedure as described in Example 8 was followed with the exception that 2 mL of the 25 wt. % solution of Monomer A in DMF, 0.9 mL of acrylic acid, and 23 mg of AIBN were used to prepare the copolymer. Infrared spectroscopy analysis of the Polymer-A4 product showed an azide peak at 2100 cm⁻¹ and amide peaks at 1630 cm⁻¹ and 1530 cm⁻¹.

### Example 11. Lacey-Layer Assembly of Poly(Ethylenimine) and Polymer-A2

Layer-by-layer (LbL) coatings were prepared using a robotic dip coater designed according to the description in Gamboa, et. al., Review of Scientific Instruments 81, 036103 (2010). The dip coater was programmed to alternate submersion of the substrate into the polycation and polyanion solutions. The dip coater was also equipped with spray nozzles for in-process washing of samples with water and separate nozzles for in-process drying of samples with compressed air. Additional features of the coater included x- and y-positioning controllers, as well as controllers for the water rinse and air-drying nozzles. The coater was placed in a safety cage.

Silicon wafer substrates (2.54×7.62 cm) were rinsed with isopropyl alcohol, dried with a stream of nitrogen gas, and then plasma cleaned for five minutes using an Atto B plasma cleaner (Diener Electronic, Ebhausen, Germany). Each cleaned substrate was mounted on the sample holder of the robotic dip coater. In the coating process, the silicon wafer substrate was quickly immersed in a 0.1 wt. % aqueous solution of branched poly(ethylenimine) (PEI) with a dwell time of 24 seconds, rinsed with deionized water, and then dried under a stream of nitrogen gas. Next, the substrate was quickly immersed in a 0.2 wt. % aqueous solution of Polymer-A2 at pH 4 with a dwell time of 24 seconds, rinsed with deionized water, and then dried. This sequence accounted for the formation of one bilayer. The sequence was repeated to prepare individual silicon wafer substrates having a total of 2, 5, or 10 coated bilayers. The total thickness of each layer-by-layer coating was measured at three randomly selected sections of a single coated substrate (n=3) using a DEKTAK XT stylus profilometer with Vision64 software (Bruker Nano, Tucson, AZ). The average total thickness results are reported in Table 1.

### Example 12. Layer-by-Layer Assembly of Poly(Ethylenimine) and Polymer-A3

The same procedure as described in Example 11 was followed with the exception that the 0.2 wt. % aqueous solution of Polymer-A2 was replaced with a 0.2 wt. % aqueous solution of Polymer-A3. The average total thickness of each layer-by-layer coating is reported in Table 1.

### Example 13. Lacey-Layer Assembly of Poly(Ethylenimine) and Polymer-A4

The same procedure as described in Example 11 was followed with the exception that the 0.2 wt. % aqueous solution of Polymer-A2 was replaced with a 0.2 wt. % aqueous solution of Polymer-A4. The average total thickness of each layer-by-layer coating is reported in Table 1.

TABLE 1

| | Average Total Thickness of Layer-by-Layer Coating Deposited | | |
|---|---|---|---|
| | on Substrate (n = 3) | | |
| Layer-by-Layer | 2 Bilayers | 5 Bilayers | 10 Bilayers |
| Assembly | Deposited | Deposited | Deposited |
| Example 11 | 13.49 ± 6.30 nm | 126.78 ± 38.24 nm | 408.56 ± 58.05 nm |
| Example 12 | 11.42 ± 3.06 nm | 127.24 ± 11.59 nm | 382.34 ± 65.04 nm |
| Example 13 | 21.36 ± 1.01 nm | 170.62 ± 10.60 nm | 322.97 ± 109.57 nm |

### Example 14. Lacey-Layer Assembly of Poly(Ethylenimine) and Polymer-A4 with Polymer Cross-Linking

A silicon wafer substrate coated with 3 bilayers of PEI and Polymer-A4 was prepared as described in Example 13. The coated substrate was placed in a 4 ounce jar that was filled with a 1 wt. % aqueous solution of glutaraldehyde. The glutaraldehyde solution was initially at about 35° C. and was allowed to slowly cool without heating while the coated substrate was immersed in the solution for 15 minutes. The substrate was removed from the jar, rinsed with deionized water, and then dried under a stream of nitrogen gas. Next, the coated substate was placed in a second 4 ounce jar filled with a 0.05 wt. % aqueous solution of 1-(3-dimethylaminopropyl)-3-ethylcarbodiimide hydrochloride (EDC). The EDC solution was initially at about 35° C. and was allowed to slowly cool without heating while the substrate was immersed in the solution for 15 minutes. The substrate was removed from the jar, rinsed with deionized water, and then dried under a stream of nitrogen gas. To test the stability of the coating, the coated substrate was first immersed in 10 mM potassium phosphate buffer (pH 7.0) for 10 minutes, rinsed with deionized water, and then dried with nitrogen gas. The coated substrate was then immersed in PMDETA for 5 minutes, rinsed with deionized water, and finally dried with nitrogen gas. Following these steps, the coating thickness was measured at three randomly selected sections of the coated substrate (n=3) using a spectroscopic ellipsometer (Model M-2000VI with WVASE32 software, J. A.

Woollam Company, Lincoln, NE). The coating was modeled as a Cauchy layer with refractive index 1.5 (An=1.5, Bn=0, Cn=0). The average thickness of the coating was 51.6±16.1 nm.

### Example 15. Preparation of an Article by Click Chemistry Cycloaddition Reaction with a Fluorescent Labeled Alkyne

Glass microscope slides (2.54×7.62 cm) were individually coated with a total of 3 bilayers of PEI and Polymer-A4 according to the procedure of Example 13. A section of the coated glass slide (2.54.cm×2.54 cm) was cut and placed in a well of a 6-well plate (VWR International, Radnor, PA). A solution of trisodium 8-prop-2-ynoxypyrene-1,3,6-trisulfonate solution in deionized water (10 mL of a 0.6 mg/mL solution) was added to the well. Next, 50 microliters of an aqueous copper sulfate solution (100 mg/mL) and 50 microliters of an aqueous sodium ascorbate solution (200 mg/mL) were added to the well. The mixture was briefly stirred using a plastic spatula and the plate was then mounted onto a low speed orbital shaker and shaken at 60 revolutions per minute (rpm) for one hour. The glass slide was removed from the plate and placed in a 4 ounce jar that was filled with deionized water. The jar was placed in an ultrasonic bath and sonicated for ten minutes. The glass slide was removed from the jar, rinsed with deionized water, and dried under a stream of nitrogen gas. The ultraviolet (UV) absorbance of the slide at 405 nm (characteristic of pyrene) was measured in transmission mode (LAMBDA 1050 UV/Vis spectrophotometer, PerkinElmer, Waltham, MA) and is reported in Table 2.

The measured absorbance was significantly higher than observed for Comparative Example A and Comparative Example B indicating that the click chemistry cycloaddition reaction had occurred and the pyrene group was covalently attached to the polymer.

### Comparative Example A

The same procedure as described in Example 15 was followed with the exception that the copper sulfate and sodium ascorbate reagents required for the click-chemistry cycloaddition reaction were not added to the well. The slide was shaken, sonicated, rinsed and dried as in Example 14. The measured UV absorbance at 405 nm is reported in Table 2. The measured absorbance was significantly lower than for Example 15 indicating that the click chemistry reaction did not occur and that the trisodium 8-prop-2-ynoxypyrene-1,3,6-trisulfonate was removed from the coated slide during the sonication and water rinse steps.

### Comparative Example B

The same procedure as described in Example 11 was followed with the exception that the 0.2 wt. % aqueous solution of Polymer-A1 was replaced with a 0.2 wt. % aqueous solution of polyacrylic acid (PAA) and glass microscope slides (2.54×7.62 cm) were used as substrates in place of silicon wafers. Each glass slide was coated to have a total of 3-bilayers of PEI and PAA. As prepared, the coating did not contain an azide functionalized polymer needed to complete the click chemistry cycloaddition reaction with trisodium 8-prop-2-ynoxypyrene-1,3,6-trisulfonate. A section of the coated glass slide (2.54×2.54 cm) was cut and placed in a well of a 6-well cell culture plate (VWR International). A solution of trisodium 8-prop-2-ynoxypyrene-1,3,6-trisulfonate solution in deionized water (10 mL of a 0.6 mg/mL solution) was added to the well. Next, 50 microliters of an aqueous copper sulfate solution (100 mg/mL) and 50 microliters of an aqueous sodium ascorbate solution (200 mg/mL) were added to the well. The mixture was briefly stirred using a plastic spatula and the plate was then mounted onto a low speed orbital shaker and shaken at 60 revolutions per minute (rpm) for one hour. The glass slide was removed from the plate and placed in a 4 ounce jar that was filled with deionized water. The jar was placed in an ultrasonic bath and sonicated for ten minutes. The glass slide was removed from the jar, rinsed with deionized water, and dried under a stream of nitrogen gas. The ultraviolet absorbance of the slide at 405 nm was measured in transmission mode (LAMBDA 1050 UV/Vis spectrophotometer, PerkinElmer) and is reported in Table 2. The measured absorbance was significantly lower than for Example 15 indicating that as expected a click chemistry reaction did not occur and that the trisodium 8-prop-2-ynoxypyrene-1,3,6-trisulfonate was removed from the coated slide during the sonication and water rinse steps.

TABLE 2

| | Absorbance at 405 nm |
|---|---|
| Example 15 | 0.2063 |
| Comparative Example A | 0.0353 |
| Comparative Example B | 0.0056 |

### Example 16. Preparation of an Article by Click Chemistry Cycloaddition Reaction with an Oligonucleotide Functionalized Alkyne

A silicon wafer substrate (2.54×7.62 cm) was coated with a total of 3 bilayers of PEI and Polymer-A4 according to the procedure of Example 13. A section of the coated substrate (2.54×2.54 cm) was cut and placed in a glass petri dish with the coated surface exposed.

A solution containing deionized water (1.48 mL), the fluorescent labeled oligonucleotide 5′-hexynyl-GCG CTG TTC ATT CGC-fluorescein-3′ (3×10⁻⁹ mol), and 7.7 microliters of an aqueous copper sulfate solution (100 mg/mL) was prepared by mixing the components in a 1.5 mL LOBIND tube (Eppendorf, Enfield, CT). An aliquot (500 microliters) of this solution was applied by pipette to the surface of the coated slide. Next, sodium ascorbate (7.7 microliters of a 200 mg/mL aqueous solution) was also applied by pipette to the surface of the coated slide. The combined liquid solution on the surface was mixed by using the pipette to withdraw and reapply the solution (3 mixing cycles of withdrawing and reapplying). The reaction was maintained for one hour and then the slide was thoroughly rinsed with deionized water and dried under a stream of nitrogen gas.

The thickness of the coating on the substrate was measured both before and after the click chemistry reaction step. The coating thickness was measured at three randomly selected sections of the coated substrate (n=3) using a spectroscopic ellipsometer (Model M-2000VI with WVASE32 software, J. A. Woollam Company). The coating was modeled as a Cauchy layer with refractive index 1.5 (An=1.5, Bn=O, Cn=O). The average thickness of the coating increased from 59.0±4.8 nm (thickness measured before the click chemistry reaction) to 67.3±0.7 nm (thickness measured after the click chemistry cycloaddition reaction with the oligonucleotide functionalized alkyne) indicating that the click chemistry reaction had occurred and the oligonucleotide was covalently attached to the polymer.

1. A monomer of Formula (I) 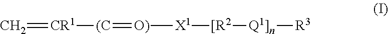
wherein
R¹ is hydrogen or methyl;
X¹ is —NH— or —O—;
n is equal to 0 or 1;
R² is an alkylene or a heteroalkylene having at least one oxygen heteroatom (i.e., ether or polyether group);
Q¹ is —(C═O)—X²— or —NH—(C═O)—X²—;
X² is —NH— or —O—; and
R³ is either
(a) an ether or polyether group terminated with an azido group; or
(b) a branched hetero-hydrocarbon group terminated with two or more azido groups, the hetero-hydrocarbon group having a branching location and comprising (i) an ether or polyether group before the branching location and (ii) an ether or polyether group in each branch after the branching location.

2. The monomer of claim 1, where in the monomer of Formula (I) is of Formula (I-A) 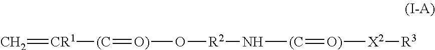
wherein
R¹ is hydrogen or methyl;
X¹ is —O—;
n is equal to 1;
R² is a heteroalkylene having at least one oxygen heteroatom (i.e., an ether or polyether group);
Q¹ is —NH—(C═O)—X²;
X² is —NH— or —O—; and
R³ is either
(a) an ether or polyether group terminated with an azido group; or
(b) a branched hetero-hydrocarbon group terminated with two or more azido groups, the hetero-hydrocarbon group having a branching location and comprising (i) an ether or polyether group before the branching location and (ii) an ether or polyether group in each branch after the branching location.

3. The monomer of claim 1, where in the monomer of Formula (I) is of Formula (I-B) 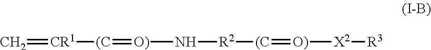
wherein
R¹ is hydrogen or methyl;
X¹ is —NH—;
n is equal to 1;
R² is an alkylene (typically —C(CH₃)₂—)
Q¹ is —(C═O)—X²;
X² is —NH— or —O—; and
R³ is either
(a) an ether or polyether group terminated with an azido group; or
(b) a branched hetero-hydrocarbon group terminated with two or more azido groups, the hetero-hydrocarbon group having a branching location and comprising (i) an ether or polyether group before the branching location and (ii) an ether or polyether group in each branch after the branching location.

4. A monomer of claim 1, where in the monomer of Formula (I) is of Formula (I-C) 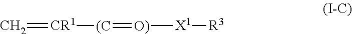
wherein
R¹ is hydrogen or methyl;
X¹ is —NH— or —O—;
n is equal to 0; and
R³ is either
(a) an ether or polyether group terminated with an azido group; or
(b) a branched hetero-hydrocarbon group terminated with two or more azido groups, the hetero-hydrocarbon group having a branching location and comprising (i) an ether or polyether group before the branching location and (ii) an ether or polyether group in each branch after the branching location.

5. The monomer of claim 1, wherein R³ is of formula —R⁴—(O—R⁴)ₘ—R⁴—N₃,
wherein each R⁴ is independently an alkylene and m is an integer in a range of 0 to 40.

6. The monomer of claim 1, wherein R³ is of formula
—R⁵—O—(R⁵—O)ₚ—R⁵—N[—R⁵—O—(R⁵—O)_{q}—R⁵—N₃]₂ where each R⁵ is independently an alkylene, p is an integer in a range of 0 to 30 and q is an integer in a range of 0 to 10.

7. The monomer of claim 1, wherein R³ is of formula
—R⁶—O—(R⁶—O)ₓ—R⁶—(C═O)—NH—CH[—R⁶—O—R⁶—(C═O)—NH—R⁶—O—(R⁶—O)_{y}—R⁶—N₃]₂ where each R⁶ is independently an alkylene, x is in integer in a range of 0 to 30 and y is an integer in a range of 0 to 10.

8. A polymer comprising a first monomeric unit derived from a monomer of Formula (I) 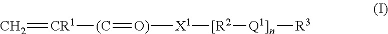
wherein
R¹ is hydrogen or methyl;
X¹ is —NH— or —O—;
n is equal to 0 or 1;
R² is an alkylene or a heteroalkylene having at least one oxygen heteroatom (i.e., ether or polyether group);
Q¹ is —(C═O)—X²— or —NH—(C═O)—X²—;
X² is —NH— or —O—; and
R³ is either
(a) an ether or polyether group terminated with an azido group; or
(b) a branched hetero-hydrocarbon group terminated with two or more azido groups, the hetero-hydrocarbon group having a branching location and comprising (i) an ether or polyether group before the branching location and (ii) an ether or polyether group in each branch after the branching location.

9. The polymer of claim 8, wherein the first monomeric unit of Formula (I) is derived from the monomers of claim 2.

10. The polymer of claim 8, further comprising a second monomeric unit comprising an acidic group or a salt thereof.

11. The polymer of claim 10, wherein the acidic group is a carboxylic acid group (—COOH), a phosphoric acid group (—O—PO₃H₂), a phosphoric acid ester group (—O—PO₃RH where R is hydrogen or alkyl), a phosphonic acid group (—OPO₃H₂), a phosphonic acid ester group (—O—PO₃RH where R is hydrogen or alkyl), sulfonic acid group (—SO₃H), or a salt thereof.

12. The polymer of claim 8, further comprising a second monomeric unit having primary amino group, secondary amino group, tertiary amino group, quaternary amino group, or a salt thereof.

13. The polymer of claim 8, further comprising a second monomeric unit comprising a hydrogen bond acceptor group or a hydrogen bond donor group.

14. The polymer of claim 8, wherein the polymer comprises 0.1 to 50 mole percent first monomeric unit based on a total weight of monomeric units in the polymer.

15. A first article comprising a substrate and a coating positioned on the substrate, wherein the coating comprises one or more layers of different polymers and wherein at least one layer comprises a first polymer comprising a first monomeric unit derived form an azido-containing monomer of Formula (I) 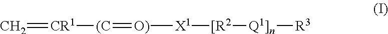
wherein
R¹ is hydrogen or methyl;
X¹ is —NH— or —O—;
n is equal to 0 or 1;
R² is an alkylene or a heteroalkylene having at least one oxygen heteroatom (i.e., ether or polyether group);
Q¹ is —(C═O)—X²— or —NH—(C═O)—X²—;
X² is —NH— or —O—; and
R³ is either
(a) an ether or polyether group terminated with an azido group; or
(b) a branched hetero-hydrocarbon group terminated with two or more azido groups, the hetero-hydrocarbon group having a branching location and comprising (i) an ether or polyether group before the branching location and (ii) an ether or polyether group in each branch after the branching location.

16. The first article of claim 15, wherein the coating comprises one or more bilayers comprising a first layer and a second layer adjacent to the first layer, wherein
a) the first layer comprises the first polymer comprising first monomeric units derived from the azido-containing monomer of Formula (I); and
b) the second layer comprises a second polymer having a monomeric unit that interacts with the first polymer electrostatically or through hydrogen bonding; and
wherein either the first layer or the second layer is adjacent to the substrate.

17. The first article of claim 16, wherein the second polymer further comprises first monomeric units derived from the azido-containing monomer of Formula (I).

18. A second article comprising a click chemistry reaction product of the first article of claim 15 with an unsaturated compound capable of undergoing a click chemistry reaction with an azido group of the first monomeric unit in the first polymer, wherein the unsaturated compound is covalently linked to the first polymer.

19. The second article of claim 18, wherein the unsaturated compound is a fluorescent-compound or a bioactive compound.

20. The second article of claim 18, or wherein the unsaturated compound has a terminal
—C≡CH group, a dibenzocyclooctyne-containing group, an unsaturated bicyclic olefinic group, or an amido group.
